(19) United States
(12) Patent Application Publication
(10) Pub. No.: US 2012/0083611 A1
(43) Pub. Date: Apr. 5, 2012

(54) PROCESS FOR PRODUCING FURFURAL

(75) Inventors: Jeroen VAN BUIJTENEN, Amsterdam (NL); Jean-Paul LANGE, Amsterdam (NL); Wouter SPIERING, Amsterdam (NL)

(73) Assignee: SHELL OIL COMPANY, Houston, TX (US)

(21) Appl. No.: 13/248,780

(22) Filed: Sep. 29, 2011

(30) Foreign Application Priority Data
Sep. 30, 2010 (EP) 10184093.2

(51) Int. Cl. C07D 307/50

(52) U.S. Cl. 549/489

(57) ABSTRACT

A process is provided for producing furfural from solid biomass by gaseous acid catalysed hydrolysis and dehydration in the presence of superheated steam and one or more acid catalysts. The solid biomass is heated to a temperature which is sufficiently high to ensure that the superheated steam and the acid catalyst or catalysts to be used remain in gaseous form during the reaction. The heated solid biomass is contacted with a gaseous stream containing superheated steam and one or more acid catalysts to produce a furfural-containing gaseous stream containing superheated steam, one or more acid catalysts and furfural. The furfural-containing gaseous stream is recirculated to bring it into further contact with the heated solid biomass to enrich the concentration of furfural.

The present application claims the benefit of EPC Application No. 10184093.2, filed Sep. 30, 2010 the entire disclosure of which is hereby incorporated by reference.

### FIELD OF THE INVENTION

The present invention relates to a process for producing furfural from biomass.

### BACKGROUND OF THE INVENTION

Furfural can be produced by acid-catalysed hydrolysis of biomass, particularly pentose-rich lignocellulosic material. Generally, furfural is converted to furfuryl alcohol for subsequent production of furan resins of use in a range of applications including corrosion-resistant materials and adhesives. Furfural is also valuable as an intermediate for the production of biofuel components

Biofuels are combustible fuels, typically derived from biological sources, which are ultimately produced from atmospheric CO₂ and can therefore be burned without net CO₂ emission. The preparation of biofuels from edible feedstock is not favoured, however, as this competes with food production and non-edible renewable feedstocks, such as lignocellulosic biomass, are therefore becoming increasingly important, both economically and environmentally.

Acid-catalysed hydrolysis of biomass leads to cleavage of covalent linkages in the cellulose, hemicellulose and lignin present and to cleavage of covalent linkages between these three components. Acids such as formic acid and acetic acid are formed together with sugars and lignin degradation products. Hemicellulose present is typically converted into five carbon sugars which are dehydrated to give furfural.

SU-1109397 describes a method of producing furfural by thermolysis of pentosan-containing feedstock at 210-220° C. in the presence of an acidic catalyst using as the heat transfer agent vapour and gases produced by the thermal breakdown of the feedstock. The pentosan-containing feedstock is heated by the circulation of the vapour and gases from bottom to top through a layer of pentosan containing feedstock that has been preheated to 50-60° C. while it is continuously turned.

In SU-1109397 a 6% solution of liquid sulphuric acid, with a boiling point at atmospheric pressure of 0.1 MPa of about 336-338° C., is used as an acidic catalyst. The process produces a furfural containing condensate at a rate of 2570 kg/hr with a content of 133.5 kg/hr furfural (i.e. a concentration of about 5 wt % furfural).

In U.S. Pat. No. 4,001,283 furfural is prepared by establishing a static bed of pentosan-containing material having a moisture content of less than 10%, introducing steam and hydrogen chloride concurrently into the bed at one end and recovering a furfural-containing mixture of gases at the other end of the bed. When hydrogen chloride begins to appear in the furfural-containing mixture of gases, the mixture of gases is conducted into a second reaction zone.

The production of furfural from raw material with high pentosan content, such as sunflower stems, corncobs or bagasse, by acid catalysis in the absence of a liquid phase is described in U.S. Pat. No. 7,173,142B. In this process as described, steam at atmospheric pressure is passed through a superheater and the stream of superheated steam is then passed through a reactor charged with comminuted raw material to strip the moisture from the charge and to heat it to the desired operating temperature. Hydrochloric acid is then introduced into the reactor by continuously dispersing it into the superheated steam via a vaporizer and the gas stream leaving the reactor is condensed. Furfural, low boiling compounds and carboxylic acids generated in the gaseous acid catalysis process are isolated from the condensate. The hydrochloric acid catalyst can be recovered as its azeotrope with water, which can be recycled by feeding it to the vaporizer in a closed circuit, thereby avoiding problems with disposal of the acid.

Although furfural can be produced in high yields by the process described in U.S. Pat. No. 7,173,142B, the furfural emerging from the reactor is highly diluted with water, acid catalyst and reaction products such as carboxylic acids. Isolating the furfural from such a reaction mixture would therefore be expected to require complicated and energy-intensive separation steps. In this process, a large amount of steam is generated and condensed per ton of furfural produced.

Thus there remains a continuing need for the development of improved methods for producing furfural from biomass.

### SUMMARY OF THE INVENTION

In one embodiment, a process is provided for producing furfural from solid biomass by gaseous acid catalysed hydrolysis and dehydration in the presence of superheated steam and at least one acid catalyst comprising the steps of

(a) heating the solid biomass to a temperature which is sufficiently high to ensure that the superheated steam and the acid catalyst or catalysts to be used remain in gaseous form during the reaction;
(b) contacting the heated solid biomass of step (a) with a gaseous stream comprising superheated steam and at least one acid catalyst to produce a furfural-containing gaseous stream comprising superheated steam, at least one acid catalyst and furfural;
(c) recirculating the furfural-containing gaseous stream to bring it into further contact with the heated solid biomass;
(d) maintaining the recirculation cycle of step (c) to enrich the concentration of furfural in the recirculating gaseous stream; and
(e) withdrawing part of the recirculating gaseous stream as a purge stream from which the furfural is separated.

### BRIEF DESCRIPTION OF THE DRAWINGS

FIG. 1 shows a schematic diagram of an embodiment of a process according to the invention.

FIGS. 2 and 3 show schematic diagrams of various methods for separating the furfural from the recirculating gaseous stream according to some embodiments of the invention.

FIG. 4 shows a phase diagram of the phase behaviour of a furfural, water, acetic acid mixture.

### DETAILED DESCRIPTION OF THE INVENTION

It has been found that an energy-efficient process for preparing furfural from biomass which is suitable for implementation on an industrial scale may be provided.

It has been found that by recirculating the gaseous stream over the heated biomass in the process according to the present invention, the amount of superheated steam that needs to be produced and condensed is significantly reduced compared to the prior art process described above, leading to considerable energy and capital investment savings.

In the process described in U.S. Pat. No. 7,173,142B, a substantial superheated steam feed is required whereas in the process of the present invention, recirculation of the gas phase decreases the need for steam input during the operation of the process. Although some of the recycled gas phase is lost in the purge stream that withdraws the products from the system, this is at least in part compensated by steam and other gaseous components produced from the biomass feedstock itself during the course of the reaction. During steady state operation of the present process, therefore, little or no additional superheated steam feed is required. This represents a significant advantage for the process of the present invention.

Furthermore, enriching the concentration of the desired furfural product in the gaseous stream phase by recirculating the gaseous stream comprising superheated steam, acid catalyst and furfural to bring it into further contact with the heated biomass facilitates the subsequent recovery of the furfural from the gaseous stream, thereby improving the energy efficiency and investment cost of the overall process.

The present process also affords the possibility of achieving furfural concentrations in the product stream that are sufficiently high for phase separation into furfural rich and water rich phases to occur, thereby facilitating the separation of furfural from water and biomass hydrolysis co-products such as acetic acid and formic acid, even when these are present in high concentrations.

Although furfural is known to undergo degradation reactions in the presence of acids under liquid phase conditions, it has surprisingly been found that recirculating furfural in a gaseous superheated steam stream in the presence of one or more acid catalysts according to the present invention does not lead to significant degradation of the furfural.

As used herein, biomass refers to an organic material of biological origin, especially to lignocellulosic material derived from plants.

Any suitable lignocellulosic material may be used in the process according to the present invention. Such lignocellulosic material may contain cellulose, hemicellulose and lignin. The hemicellulose contains pentosans which can be converted into furfural.

In an embodiment of the process, solid biomass rich in pentosans is preferred. Preferably the solid biomass used as a feed in the process of the invention comprises equal to or more than 1 wt % of pentosans, more preferably equal to or more than 5 wt % pentosan, still more preferably equal to or more than 10 wt % pentosans most preferably equal to or more than 15 wt % pentosans. Although there is no upper limit, for practical purposes, the solid biomass used as a feed in the process of the invention may comprise equal to or less than 90 wt % pentosans, preferably equal to or less than 60 wt % pentosans, more preferably equal to or less than 40 wt % pentosans and most preferably equal to or less than 35 wt % pentosans.

The solid biomass may be obtained from a variety of plants and plant materials including agricultural wastes, forestry wastes and sugar processing residues. Examples of suitable solid biomass include agricultural wastes such as corn stover, soybean stover, corn cobs, rice straw, rice hulls, oat hulls, corn fibre, cereal straws such as wheat, barley, rye and oat straw; grasses; forestry products such as wood and wood-related materials such as sawdust; waste paper; sugar processing residues such as sugarcane bagasse and beet pulp; or mixtures thereof.

The solid biomass may be comminuted into small pieces in order to facilitate hydrolysis. Conveniently, the solid biomass is comminuted into pieces of average length of 0.5 to 3 cm.

The solid biomass used as starting material is suitably heated in step (a) to a temperature of from 100° C. to 300° C. to produce a heated solid biomass. In one embodiment, the solid biomass used as starting material is heated to a temperature of from 120° C. to 250° C.

Step (a) can be carried out at a wide variety of pressures. Preferably step (a) can be carried out at a pressure in the range from 0.01 to 1 MPa (0.1 to 10 bar), more preferably in the range from 0.05 to 0.5 MPa (0.5 to 5 bar), most preferably at a pressure of about 0.1 MPa (about 1 bar).

The temperature at which the heated solid biomass is contacted with the gaseous stream comprising superheated steam and one or more acid catalysts in step (b) is conveniently sufficiently high that the steam and acid catalyst or catalysts remain in vapour form at the operating pressure under which the reaction is performed.

In one embodiment, the heated solid biomass is contacted with the gaseous stream comprising superheated steam and one or more acid catalysts when the heated solid biomass is at a temperature of from 120° C. to 250° C., for example from 140° C. to 190° C.

Step (b) can be carried out at a wide variety of pressures. Preferably step (b) can be carried out at a pressure in the range from 0.01 to 1 MPa (0.1 to 10 bar), more preferably in the range from 0.05 to 0.5 MPa (0.5 to 5 bar), most preferably at a pressure of about 0.1 MPa (about 1 bar).

The heated solid biomass will be contacted with the gaseous stream comprising superheated steam and the acid catalyst or catalysts for a time period sufficient to achieve hemicellulose hydrolysis. This time period may for example range from 0.5 hour to 20 hours, for example from 2 hours to 10 hours.

By superheated steam is herein preferably understood steam that is above its dew point, preferably equal to or more than 5° C. above its dew point, more preferably equal to or more than 30° C. above its dewpoint, and most preferably equal to or more than 50° C. above its dewpoint. The person skilled in the art will understand that the dewpoint will be the dewpoint under the reaction conditions applied and may depend on for example the pressure.

In one embodiment, the gaseous stream comprising superheated steam and one or more acid catalysts is prepared by dispersing an aqueous solution of the acid catalyst or catalysts into the superheated steam. Preferably the acid catalyst or catalysts are dispersed in an amount sufficient to produce a gaseous stream having the desired acid concentration. It will be appreciated that the concentration of catalyst will depend on the particular catalyst employed. Conveniently, where the catalyst is hydrochloric acid, the concentration of the acid catalyst is controlled at an amount of from 0.5 to 5 wt % based on the total weight of the catalyst and the water present.

The gaseous stream may comprise one or more acid catalyst(s). Conveniently, these one or more acid catalyst(s) is/are in the gaseous form at the reaction conditions applied.

By an acid catalyst in the gaseous form is herein preferably understood that equal to or more than 80 mol %, more preferably equal to or more than 90 mol %, still more preferably equal to or more than 95 mol %, even more preferably equal to or more than 97 mol %, and most preferably equal to or more than 99 mol %, based on the total amount of moles of the acid catalyst present in step (b), is in the gaseous form.

Conveniently the one or more acid catalyst(s) may comprise one or more acid(s) that have a boiling point below the reaction temperature at the reaction pressure applied. Preferably the one or more acid catalyst(s) comprise one or more acid(s) that have a boiling point at about 0.1 MPa (1 bar) of equal to or less than 250° C., even more preferably equal to or less than 140° C. and most preferably equal to or less than 110° C.

If the one or more acid catalyst(s) are present as an azeotrope with water, the aqueous azeotrope of the one or more acid catalyst(s) may conveniently have a boiling point below the reaction temperature at the reaction pressure applied. Preferably this aqueous azeotrope of one or more acid catalyst(s) may have a boiling point at about 0.1 MPa (1 bar) of equal to or less than 250° C., even more preferably equal to or less than 140° C. and most preferably equal to or less than 110° C.

In one embodiment, an acid catalyst for use in the process of the invention is a volatile Brønsted acid or Lewis acid. Any known volatile acid catalyst, or mixture of acid catalysts, conventional in the art may be employed provided that the acid, or acids, are sufficiently strong to mediate depolymerisation and dehydration of the biomass material, and are in gaseous form under the operating conditions employed.

In one embodiment, a Brønsted acid catalyst for use in the process of the invention is hydrochloric acid. In another embodiment, a Lewis acid catalyst such as for example sulphurdioxide (SO₂), boron trifluoride or trifluoroacetic acid may be used in the process of the invention.

In a preferred embodiment the one or more acid catalyst(s) comprise one or more acid(s) having a pKa at 25° C. of equal to or less than 3, more preferably of equal to or less than 2, still more preferably of equal to or less than 1 and most preferably of equal to or less than 0. For example hydrochloric acid has a pKa at 25° C. of less than 0. Such pKa values may for example be found according to E. P. Serjeant and B. Dempsey (eds.), Ionization Constants of Organic Acids in Solution, IUPAC Chemical Data Series No. 23, Pergamon Press, Oxford, UK, 1979.

In a preferred embodiment the one or more acid catalyst(s) comprise one or more acid(s) chosen from the group consisting of hydrochloric acid, sulphurdioxide (SO₂), boron trifluoride or trifluoroacetic acid. In an especially preferred embodiment the one or more acid catalyst(s) comprise hydrochloric acid.

Steps (b) and (c) of the process of the invention may be carried out in a reactor. The reactor may be configured in any known way suitable to allow solid-gas contact, for example the reactor may be configured such that the solid biomass and gaseous stream flow concurrently with or countercurrently against each other.

In one embodiment, the reactor is a moving bed reactor and the solid biomass may move in a downflow direction. Alternatively, the reactor may be a fluidized bed reactor in which the solid biomass can move in an upflow direction or a conveyor reactor having a conveyor screw or conveyor belt configured to move the solid biomass in horizontal, upflow or downflow direction.

In steps (b) and (c), at least part of the solid biomass is hydrolysed to produce furfural. Other biomass hydrolysis products such as acetic acid and formic acid may also be produced in commercially useful amounts. Production of such other biomass hydrolysis products of interest forms another aspect of the present invention.

Suitably the process according to the invention is performed at atmospheric pressure, although higher or lower pressures are also contemplated.

Recirculating the furfural containing gaseous stream produced in step (b) by bringing it into further contact with the solid biomass in steps (c) and (d) has the effect of enriching the concentration of furfural in the gaseous stream. In this way, furfural concentrations of up to 50 wt %, conveniently of from 10 to 30 wt % may be built up. Increasing the concentration of furfural in the gaseous product stream is advantageous as it enables phase separation of the product stream into furfural rich and water rich phases to occur.

In one embodiment, if the concentration of furfural in the purge stream is sufficiently high, phase separation may be used to recover the furfural. Condensation of the purge stream leads to the formation of two separate liquid phases, an organic phase rich in furfural and an aqueous phase. The organic phase will still contain some water and other by-products and this may be removed by distillation, pervaporation or adsorption, for example. The aqueous phase contains the one or more acid catalysts, acetic acid and will also contain some furfural; distillation recovers the furfural present as a water-furfural azeotrope which can be returned to the condenser so that more of the furfural can be extracted. The acid catalyst or catalysts can be recovered from the remaining water/acid catalyst/acetic acid and can then be reused.

If the furfural concentration in the purge stream is not high enough for phase separation to occur, furfural can be recovered by distillation of a furfural-water azeotrope from the purge stream and subsequent liquid-liquid separation of the azeotrope into furfural-rich and a water-rich stream upon condensation.

After the desired amount of furfural is obtained from the solid biomass, the remaining biomass, hereafter referred to as biomass residue, can be retrieved from the reactor. The biomass residue may be retrieved from the reactor in a batch-wise or continuous manner.

Preferably this biomass residue is not discarded but further processed and/or used for a subsequent purpose. To improve handling of the biomass residue in any subsequent use or further processing, the biomass residue may for example be densified. Any densification technique known by the skilled person to be suitable for this purpose may be used, including for example pelletization techniques.

In one preferred embodiment of the invention the, optionally densified, biomass residue produced in the process of the invention may be used as a source for heat and/or power generation.

In another preferred embodiment of the invention the, optionally densified, biomass residue produced in the process of the invention may be used as a feed in a gasification process to prepare synthesis gas. This synthesis gas can be used as a source of hydrogen and/or as a source of power and/or to prepare valuable chemicals and/or automotive fuels in a Fisher-Tropsch process.

In another preferred embodiment of the invention the, optionally densified, biomass residue produced in the process of the invention can be converted to obtain one or more biofuel components and/or biochemicals and/or one or more intermediates for the production of such biofuel components and/or biochemicals. Such conversion may be carried out using any method known by the skilled person in the art to be suitable for this purpose. The conversion may for example include hydrolysis, hydrogenolysis, pyrolysis, liquefaction, hydroliquefaction and/or any combination thereof. When converting the biomass residue by means of for example hydrolysis, hydrogenolysis, pyrolysis, liquefaction, hydroliquefaction and/or any combination thereof, a wide range of products may be obtained. The products of the conversion of the biomass residue may for example include sugars (for example glucose, xylose); anhydrosugars (for example levoglucosan); sugar alcohols and polyols (for example sorbitol, mannitol, isosorbide, ethane/propane diols, glycerols); furans (for example hydroxymethylfurfural); monolignols (for example coniferyl alcohol, sinapyl alcohol and paracoumaryl alcohol); acids (for example levulinic acid); alcohols (for example ethanol, butanol); alkanes; derivatives, oligomers and/or co-oligomers of all the before mentioned and/or mixtures of all the before mentioned. The specific products may be isolated from a total product obtained after conversion by any method know to the skilled person to be suitable for this purpose. Such methods may include fractionation, phase separation, and/or extraction. The, optionally isolated, products may be useful as a component in a biofuel or biochemical or as an intermediate in the production of a biofuel and/or biochemical. For example the biomass residue may be subjected to hydrolysis and/or liquefaction to obtain sugars, levulinic acid and/or hydroxymethylfurfural. The sugars may for example in turn be dehydrated to prepare levulinic acid or hydroxymethylfurfural, and/or hydrogenolysized to prepare oxygenated precursors for biofuels and/or fermented to prepare alcohols such as ethanol or butanol, carboxylic acids, esters or hydrocarbons.

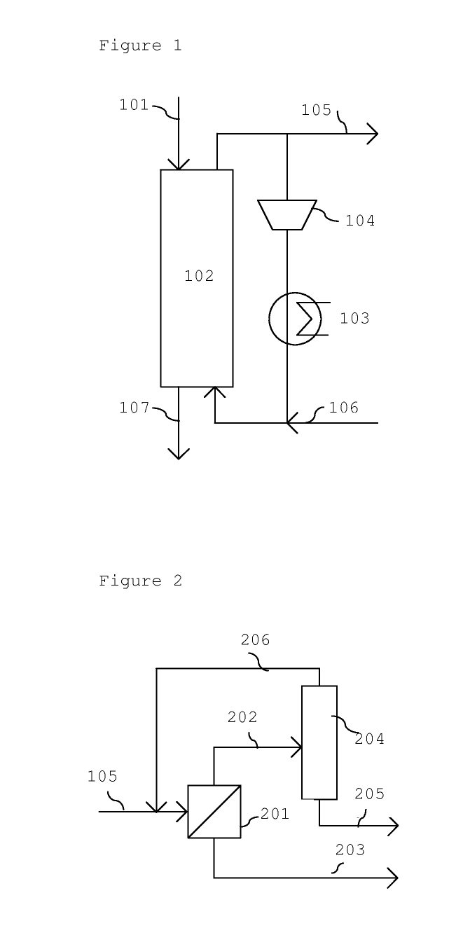

FIG. 1 shows a process scheme for an embodiment of the process according to the invention.

In the embodiment shown, solid biomass (101) is supplied to hydrolysis reactor (102). A gaseous stream comprising superheated steam, from heat exchanger (103) and acid catalyst are recirculated through reactor (102) by a blower (104). In the embodiment shown, the solid biomass flows in a downflow direction through the reactor, which is a moving bed reactor, and the gaseous stream flows countercurrently to the biomass. Part of the furfural containing recirculating gaseous stream is withdrawn from the reactor in a purge gas stream (105) for optional transfer to a liquid/liquid separator (not shown). Make-up acid catalyst feed (106) is supplied to the reactor to replace the acid catalyst withdrawn with the purge gas stream. Solid biomass residue (107) is withdrawn from the hydrolysis reactor for optional further treatment.

Schemes for the recovery of furfural from the gas phase purge (105) are shown in FIGS. 2 and 3.

FIG. 2 illustrates a downstream separation stage which employs phase-separation to recover the furfural. The withdrawn furfural containing purge gas stream (105) is fed via a condenser (not shown) where the gas stream is condensed into a liquid/liquid separator (201) and separates into an aqueous phase (202) (comprising mainly water and acid catalyst with some furfural and acetic acid also) and an organic phase (203) (comprising mainly furfural but with some water and acetic acid). The aqueous phase (202) is transferred to a distillation column (204) where it separates into water/acetic acid/acid catalyst (205) and a furfural/water azeotrope (206) which is returned to the liquid/liquid separator (201) so that more of the furfural can be extracted.

FIG. 3 shows an alternative embodiment which does not involve natural liquid-liquid phase separation of the purge stream. Here, the withdrawn furfural containing purge gas stream (105) is fed via a condenser (not shown) where the gas stream is condensed into a distillation column (301). Water/acetic acid/acid catalyst is separated off (302) and furfural in the form of a furfural/water azeotrope (303) is transferred to a condenser/separator (304) where it is separated into an organic phase (305) mainly comprising furfural (with some water and acetic acid) and an aqueous phase (306) (mainly water with some furfural) which is returned to the distillation column for further separation and recovery of the furfural.

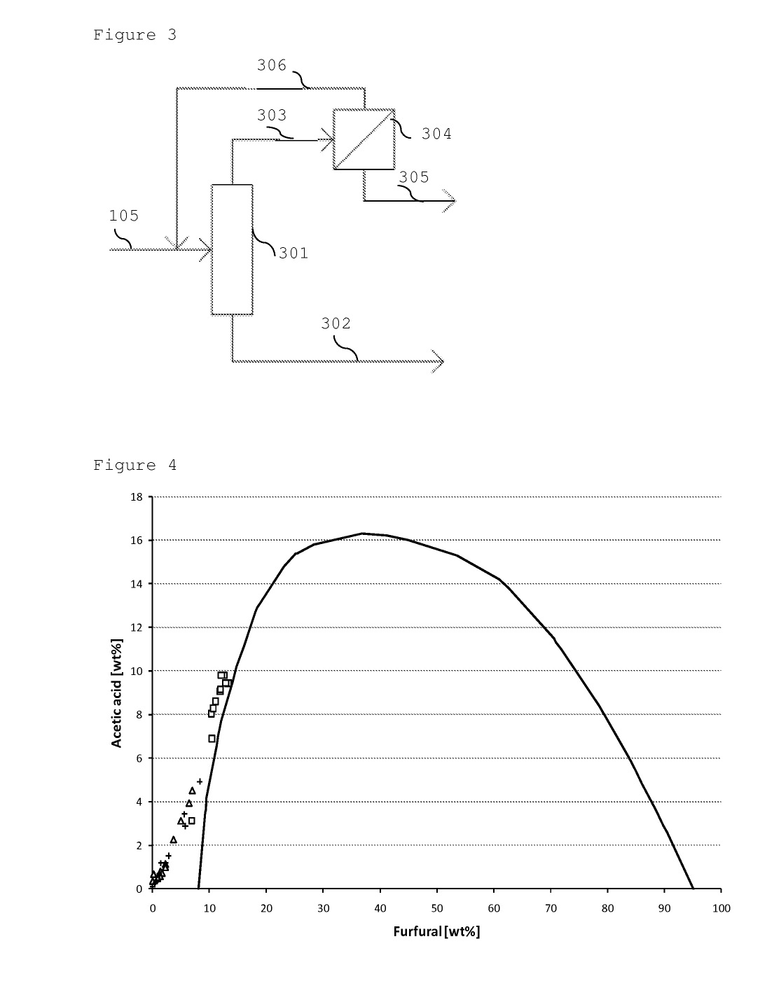

FIG. 4 shows a phase diagram for furfural/water/acetic acid mixtures obtained at 25° C. (Heric et al, Can. J. Chem. Eng, 38, 46-48, 1960; Heric et al, J. Chem Eng. Data, 5(3), 272-274, 1960). These conditions are similar to the conditions that apply for the condensates obtained in the process of the present invention. In the region underneath the curve, liquid-liquid phase separation occurs naturally. The experimental results for Example 1 (1 wt % HCl, 158 C, +) and Example 2 (1 wt % HCl, 158 C, Δ) from Table 1 below, that are shown on the graph, demonstrate that furfural concentrations obtained in the absence of recycling are generally too low to give liquid-liquid phase separation. However, it is also clear from the phase diagram that a modest increase in furfural concentration would lead to liquid-liquid phase separation. This is demonstrated by the experimental results for Example 12 (1 wt % HCl, 10 wt % furfural, 6 wt % acetic acid, 160 C, □), an experiment with furfural and acetic acid in the superheated gaseous feed at concentrations that are representative for recycle after once through operation. The composition of the samples obtained reaches the region for which phase separation can be expected and in several samples a separate organic phase was indeed observed.

Throughout the description and claims of this specification, the words “comprise” and “contain” and variations of the words, for example “comprising” and “comprises”, mean “including but not limited to”, and do not exclude other moieties, additives, components, integers or steps. The singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples.

Generally speaking the invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the invention are to be understood to be applicable to any other aspect, embodiment or example described herein unless incompatible therewith.

Moreover unless stated otherwise, any feature disclosed herein may be replaced by an alternative feature serving the same or a similar purpose.

### EXAMPLES

The invention will now be further illustrated by means of the following non-limiting examples.

### General Experimental Procedure

In the examples described below, the following general experimental method was employed:

A stationary bed of pre-dried sugarcane bagasse was placed in a glass column equipped with a heating mantle. The bagasse was heated to the desired operating temperature in the range of 150° C.-210° C. at ambient pressure (about 0.1 MPa, i.e. about 1 bar). When the bed reached a temperature in the range 120° C.-130° C., acid-containing superheated steam was introduced by pumping a solution of acid in water through a heated glass line at 225° C. Vapours were condensed and analyzed with GC-FID (samples collected every 30-60 min). The reaction was terminated after approximately 5-6 h.

### Examples 1-17

Experiments were performed using hydrochloric acid (Examples 1-12), formic acid (Example 13) and acetic acid (Examples 14-16) as acid catalyst and with no acid catalyst as a control (Example 17).

The experimental conditions employed (temperature, acid catalyst, acid concentration, flow rate and reaction time) for each experiment are recorded in table 1 below together with the final yields of furfural (FFR) and acetic acid (AA) and the highest concentrations of furfural and acetic acid achieved in the samples for each experiment. The final yields of furfural (FFR) and acetic acid (AA) are calculated as a percentage of the theoretical value obtained after a 100% conversion of hemicellulose into these products. The mass balance is also recorded.

TABLE 1
Experimental results obtained using various acid catalysts

| | | | | | | Final | Final | Highest | Highest | |
|---|---|---|---|---|---|---|---|---|---|---|
| | | | Acid | | | Yield | Yield | conc. | conc. | Mass |
| | Temp | Acid | conc. | Flow rate | Reaction | FFR | AA | FFR | AA | balance |
| Ex. | (° C.) | cat. | (wt %) | (ml/min) | time (h) | (mol %) | (mol %) | (wt %) | (wt %) | (%) |
| 1 | 158 | HCl | 1 | 0.17 | 6.3 | 44% | 86% | 5.4% | 3.0% | 94% |
| 2 | 158 | HCl | 1 | 0.17 | 6.2 | 53% | 107% | 7.0% | 4.5% | 96% |
| 3^{a} | 158 | HCl | 1 | 0.17 | 6.0 | 44% | 85% | 8.3% | 4.9% | 94% |
| 4 | 160 | HCl | 1 | 0.34 | 5.6 | 41% | 71% | 4.6% | 2.0% | 90% |
| 5 | 160 | HCl | 0.33 | 0.17 | 7.1 | 36% | 73% | 2.7% | 1.8% | 96% |
| 6 | 158 | HCl | 3 | 0.17 | 5.9 | 47% | 87% | 12.3% | 6.8% | 92% |
| 7 | 180 | HCl | 1 | 0.17 | 6.6 | 35% | 94% | 6.2% | 4.6% | 93% |
| 8 | 180 | HCl | 1 | 0.17 | 5.5 | 35% | 85% | 5.3% | 3.1% | 95% |
| 9 | 182 | HCl | 1 | 0.06 | 6.8 | 20% | 71% | 6.0% | 4.2% | 76% |
| 10 | 205 | HCl | 1 | 0.17 | 4.8 | 18% | 96% | 3.6% | 2.4% | 82% |
| 11^{b} | 170 | HCl | 1 | 0.17 | 6.1 | 29% | 62% | 3.1% | 1.2% | 83% |
| 12^{c} | 160 | HCl | 1 | 0.17 | 7.1 | 37% | 76% | 13.3% | 9.4% | 97% |
| 13 | 158 | FA | 20 | 0.17 | 6.0 | 2% | 28% | 0.2% | 0.5% | 94% |
| 14 | 164 | AA | 20 | 0.20 | 5.9 | 0% | n.a. | n.a. | n.a. | 81% |
| 15 | 188 | AA | 20 | 0.20 | 6.3 | 0% | n.a. | 0.2% | n.a. | 92% |
| 16 | 208 | AA | 20 | 0.20 | 6.0 | 1% | n.a. | 0.3% | n.a. | 94% |
| 17 | 180 | none | n.a. | 0.17 | 5.9 | 0% | 18% | 0.1% | 0.7% | 91% |

^{a}Experiment with ground bagasse (0.6 mm < particle size < 0.85 mm)
^{b}Experiment with 3 wt % 5-MeFL in the superheated steam
^{c}Experiment with 10 wt % FFR and 6 wt % AA in the superheated steam
Theoretical yields of furfural and acetic acid are 14.8 wt % and 4.4 wt % on dry biomass respectively.

From the results presented in table 1 above it can be seen that the strong acid hydrochloric acid is particularly suitable for use as a catalyst according to the invention. Formic acid and acetic acid, both of which are weak acids, were found to be much less effective as catalysts for use in producing furfural.

### Example 11 with 5-Methylfurfural in the Superheated Gaseous Feed

In the experiment of Example 11, 5-methylfurfural was added to the superheated steam feed, to assist in determining the amount of furfural that was added during the circulation. In this case, the stationary bed of pre-dried sugarcane bagasse was heated to 170° C. at ambient pressure and the superheated gas-phase (steam containing 1 wt % HCl and 3 wt % 5-methylfurfural) was introduced when the bed reached a temperature of 120-130° C.

In contrast to the homogeneous feedstock, several of the collected samples show phase separation between an aqueous phase and a furfural/5-methylfurfuryl-rich phase. Yields for furfural and acetic acid are respectively 29 mol % on pentosan and 62 mol % on acetyl groups. The 5-Methylfurfural recovered in the condensate was on average 96% of the feed concentration, demonstrating the potential for build-up of the concentration of furfural-like components by recycling without significant decomposition.

In addition this example shows that an additional 3.1 wt % furfural (as illustrated in table 1) could be taking up by the gaseous stream, that already contained furfural-like components.

### Example 12 with Furfural/Acetic Acid in the Superheated Gas Feed

In the experiment of Example 12, the bagasse bed was treated with a superheated steam feed containing 10 wt % furfural, 6 wt % acetic acid and 1 wt % HCl, to simulate recirculating a gaseous stream comprising superheated steam, furfural and one or more acid catalyst(s). In contrast to the homogeneous feedstock, several of the collected samples show phase separation between an aqueous phase and a furfural-rich phase. Yields for furfural and acetic acid were respectively 37 mol % on pentosan and 76 mol % on acetyl groups.

These results demonstrate that furfural does not undergo significant decomposition even during prolonged exposure to the reaction conditions employed, again supporting the potential for increasing furfural concentration by recycling. It also demonstrates that recycling in order to build up the furfural concentration in the superheated gas phase does indeed enable liquid-liquid phase separation. Although the concentration of furfural is slightly below that at which phase separation would be expected from the phase diagram shown in FIG. 4, this can be explained by the difference in temperature of the actual fractions and by the presence of HCl and other minor by-products.

We claim:

1. A process for producing furfural from solid biomass in the presence of superheated steam and at least one acid catalyst comprising the steps of
(a) heating the solid biomass to a temperature which is sufficiently high to ensure that the superheated steam and the acid catalyst or catalysts to be used remain in gaseous form during the reaction;
(b) contacting the heated solid biomass of step (a) with a gaseous stream comprising superheated steam and at least one acid catalyst to produce a furfural-containing gaseous stream comprising superheated steam, at least one acid catalyst and furfural;
(c) recirculating the furfural-containing gaseous stream to bring it into further contact with the heated solid biomass;
(d) maintaining the recirculation cycle of step (c) to enrich the concentration of furfural in the recirculating gaseous stream; and
(e) withdrawing part of the recirculating gaseous stream as a purge stream from which the furfural is separated.

2. The process of claim 1 wherein the solid biomass comprises equal to or more than 5 wt % pentosans.

3. The process of claim 1 wherein the heated solid biomass of step (a) is contacted with the gaseous stream comprising superheated steam and the acid catalyst at a temperature in the range of from 120° C. to 250° C.

4. The process of claim 3 wherein the heated solid biomass of step (a) is contacted with the gaseous stream comprising superheated steam and the acid catalyst at a temperature in the range of 140° C. to 190° C.

5. The process of claim 1 wherein the acid catalyst comprises hydrochloric acid.

6. The process of claim 5 wherein the concentration of the acid catalyst is controlled at an amount of from 0.5 wt % to 5 wt % based on the weight of catalyst and water present.

7. The process of claim 1 wherein the heated solid biomass is contacted with the gaseous stream comprising superheated steam and the acid catalyst in a moving bed reactor.

8. The process of claim 1 wherein the gaseous stream produced in step (b) further comprises acetic acid and/or formic acid.

9. The process of claim 1 wherein the concentration of furfural in the recirculating gaseous stream of step (d) is built up to equal to or less than 50 wt %.

10. The process of claim 3 wherein the concentration of furfural in the recirculating gaseous stream of step (d) is built up to equal to or less than 50 wt %.

11. The process of claim 1 wherein a furfural-rich phase is separated from the withdrawn purge stream of step (e) by liquid-liquid phase separation.

12. The process of claim 1 wherein the withdrawn purge stream is subjected to a further distillation step prior to separating the furfural.

13. The process of claim 1 wherein further obtaining a biomass residue and using said biomass residue as a source of heat or power.

14. The process of claim 1 wherein further obtaining a biomass residue and using said biomass residue as a feed for a gasification process.

15. The process of claim 1 wherein further obtaining a biomass residue and converting said biomass residue to obtain biofuels components and/or biochemicals and/or one or more intermediates for the production of biofuel components and/or biochemicals.

16. The process of claim 13 wherein the biomass residue is densified.
